SEQUENCE LISTING (10 sequences)

SEQ ID NO: 1 - DNA, 1479 bp, Homo sapiens
  CDS (1)..(1479)
atggcggccggcgaaaggagctggtgcctgtgcaagttgttgaggtttttttattcattattcttccctgggctcattgtatgtggaactttatgtgtgtgtttggtcattgtcctttggggaatcagactgctgctacagagaaagaaaaaattagtgtcaactagcaaaaatgggaaaaatcaaatggtgattgcattttttcatccatactgcaatgctggtggaggaggagaaagagttttatggtgtgctttaagagccctgcagaaaaagtatcctgaagcagtttatgttgtttataccggcgatgttaatgtcaacggtcaacagatactagaaggtgctttcagaagatttaacatcagattaattcacccagtgcagtttgtttttttaaggaaacgctatcttgtggaagattcactgtatcctcacttcacactgctgggccaaagtctaggatccatttttcttggctgggaagctctaatgcagtgtgttcctgatgtttacattgattcaatgggatacgcttttacgcttcctctgtttaagtatatagggggttgccaagttggaagctatgttcattatcctactatcagcaccgacatgctctctgtagtgaagaatcaaaatattggatttaataatgcagccttcattaccaggaatccttttctcagcaaagtaaagctcatctactactatttatttgcttttatttatggacttgttggttcttgcagtgatgtagtcatggtcaattcttcttggacactaaaccatattctctcactatggaaagttgggaattgcactaacattgtttatccaccttgtgatgtgcagacatttctggacattcccttacatgagaaaaagatgaccccaggacatttgctggtttctgttggccagtttaggccggaaaagaatcatccattgcagatcagagcctttgctaaattgctgaataagaagatggttgagtcacctccttcgcttaaacttgtcctcattggaggttgtcgtaacaaagatgatgaacttagggtaaaccaactgagaaggctgtctgaggatttaggagttcaagaatatgtggaatttaaaataaacattccatttgatgaattaaagaattatttgtctgaagcaacaattggtctgcataccatgtggaacgagcattttgggattggagttgtggagtgtatggcagctggcacaattatccttgcacacaattcggggggcccaaagcttgacattgtggttcctcacgaaggagatataactggctttctggctgagagtgaagaagactatgctgaaactatcgctcacattctttccatgtctgcagaaaagagactccaaatcagaaaaagtgctcgtgcatctgtaagcagattctctgatcaggaatttgaagtgacattcctatcatctgtggaaaagttatttaagtaa

SEQ ID NO: 2 - protein, 492 aa, Homo sapiens
MAAGERSWCLCKLLRFFYSLFFPGLIVCGTLCVCLVIVLWGIRLLLQRKKKLVSTSKNGKNQMVIAFFHPYCNAGGGGERVLWCALRALQKKYPEAVYVVYTGDVNVNGQQILEGAFRRFNIRLIHPVQFVFLRKRYLVEDSLYPHFTLLGQSLGSIFLGWEALMQCVPDVYIDSMGYAFTLPLFKYIGGCQVGSYVHYPTISTDMLSVVKNQNIGFNNAAFITRNPFLSKVKLIYYYLFAFIYGLVGSCSDVVMVNSSWTLNHILSLWKVGNCTNIVYPPCDVQTFLDIPLHEKKMTPGHLLVSVGQFRPEKNHPLQIRAFAKLLNKKMVESPPSLKLVLIGGCRNKDDELRVNQLRRLSEDLGVQEYVEFKINIPFDELKNYLSEATIGLHTMWNEHFGIGVVECMAAGTIILAHNSGGPKLDIVVPHEGDITGFLAESEEDYAETIAHILSMSAEKRLQIRKSARASVSRFSDQEFEVTFLSSVEKLFK

SEQ ID NO: 3 - DNA, 1581 bp, Homo sapiens
  CDS (1)..(1581)
atggcggcgctcacaattgccacgggtactggcaattggttttcggctttggcgctcggggtgactcttctcaaatgccttctcatccccacataccattccacagattttgaagtacaccgaaactggcttgctatcactcacagtttgccaatatcacagtggtattatgaggcaacttcagagtggacgttggattacccccctttctttgcatggtttgagtatatcctgtcacatgttgccaaatattttgatcaagaaatgctgaatgtccataatttgaattactccagctcaaggaccttacttttccagagattttccgtcatctttatggatgtactctttgtgtatgctgtccgtgagtgctgtaaatgcattgatggaaaaaaagtgggtaaagaacttacagaaaagccaaaatttattctgtcggtattacttctgtggaacttcgggttattaattgtggaccatattcattttcagtacaatggctttttatttggattaatgctactctccattgcacgattatttcagaaaaggcatatggaaggagcatttctctttgctgttctcctacatttcaagcatatctacctctatgtagcaccagcttatggtgtatatctgctgcgatcctactgtttcactgcaaataaaccagatgggtctattcgatggaagagtttcagctttgttcgtgttatttccctgggactggttgttttcttagtttctgctctttcattgggtcctttcctggccttgaatcagctgcctcaagtcttttcccgactctttcctttcaagaggggcctctgtcatgcatattgggctccaaacttctgggctttgtacaatgctttggacaaagtgctgtctgtcatcggtttgaaattgaaatttcttgatcccaacaatattcccaaggcctcaatgacaagtggtttggttcagcagttccaacacacagtccttccctcagtgactcccttggcaaccctcatctgcacactgattgccatattgccctctattttctgtctttggtttaaaccccaagggcccagaggctttctccgatgtctaactctttgtgccttgagctcctttatgtttgggtggcatgttcatgaaaaagccatacttctagcaattctcccaatgagccttttgtctgtgggaaaagcaggagacgcttcgatttttctgattctgaccacaacaggacattattccctctttcctctgctcttcactgcaccagaacttcccattaaaatcttactcatgttactattcaccatatatagtatttcgtcactgaagactttattcagaaaagaaaaacctctttttaattggatggaaactttctacctgcttggcctggggcctctggaagtctgctgtgaatttgtattccctttcacctcctggaaggtgaagtaccccttcatccctttgttactaacctcagtgtattgtgcagtaggcatcacatatgcttggttcaaactgtatgtttcagtattgattgactctgctattggcaagacaaagaaacaatga

SEQ ID NO: 4 - protein, 526 aa, Homo sapiens
MAALTIATGTGNWFSALALGVTLLKCLLIPTYHSTDFEVHRNWLAITHSLPISQWYYEATSEWTLDYPPFFAWFEYILSHVAKYFDQEMLNVHNLNYSSSRTLLFQRFSVIFMDVLFVYAVRECCKCIDGKKVGKELTEKPKFILSVLLLWNFGLLIVDHIHFQYNGFLFGLMLLSIARLFQKRHMEGAFLFAVLLHFKHIYLYVAPAYGVYLLRSYCFTANKPDGSIRWKSFSFVRVISLGLVVFLVSALSLGPFLALNQLPQVFSRLFPFKRGLCHAYWAPNFWALYNALDKVLSVIGLKLKFLDPNNIPKASMTSGLVQQFQHTVLPSVTPLATLICTLIAILPSIFCLWFKPQGPRGFLRCLTLCALSSFMFGWHVHEKAILLAILPMSLLSVGKAGDASIFLILTTTGHYSLFPLLFTAPELPIKILLMLLFTIYSISSLKTLFRKEKPLFNWMETFYLLGLGPLEVCCEFVFPFTSWKVKYPFIPLLLTSVYCAVGITYAWFKLYVSVLIDSAIGKTKKQ

SEQ ID NO: 5 - DNA, 1857 bp, Homo sapiens
  CDS (1)..(1857)
atggctagtcgaggggctcggcagcgcctgaagggcagcggggccagcagtggggatacggccccggctgcggacaagctgcgggagctgctgggcagccgagaggcgggcggcgcggagcaccggaccgagttatctgggaacaaagcaggacaagtctgggcacctgaaggatctactgctttcaagtgtctgctttcagcaaggttatgtgctgctctcctgagcaacatctctgactgtgatgaaacattcaactactgggagccaacacactacctcatctatggggaagggtttcagacttgggaatattccccagcatatgccattcgctcctatgcttacctgttgcttcatgcctggccagctgcatttcatgcaagaattctacaaactaataagattcttgtgttttactttttgcgatgtcttctggcttttgtgagctgtatttgtgaactttacttttacaaggctgtgtgcaagaagtttgggttgcacgtgagtcgaatgatgctagccttcttggttctcagcactggcatgttttgctcatcatcagcattccttcctagtagcttctgtatgtacactacgttgatagccatgactggatggtatatggacaagacttccattgctgtgctgggagtagcagctggggctatcttaggctggccattcagtgcagctcttggtttacccattgcctttgatttgctggtcatgaaacacaggtggaagagtttctttcattggtcgctgatggccctcatactatttctggtgcctgtggtggtcattgacagctactattatgggaagttggtgattgcaccactcaacattgttttgtataatatctttactcctcatggacctgatctttatggtacagaaccctggtatttctatttaattaatggatttctgaatttcaatgtagcctttgctttggctctcctagtcctaccactgacttctcttatggaatacctgctgcagagatttcatgttcagaatttaggccacccgtattggcttaccttggctccaatgtatatttggtttataattttcttcatccagcctcacaaagaggagagatttcttttccctgtgtatccacttatatgtctctgtggtgctgtggctctctctgcacttcagcacagttttctgtacttccagaaatgttaccactttgtgtttcaacgatatcgcctggagcactatactgtgacatcgaattggctggcattaggaactgtcttcctgtttgggctcttgtcattttctcgctctgtggcactgttcagaggatatcacgggccccttgatttgtatccagaattttaccgaattgctacagacccaaccatccacactgtcccagaaggcagacctgtgaatgtctgtgtgggaaaagagtggtatcgatttcccagcagcttccttcttcctgacaattggcagcttcagttcattccatcagagttcagaggtcagttaccaaaaccttttgcagaaggacctctggccacccggattgttcctactgacatgaatgaccagaatctagaagagccatccagatatattgatatcagtaaatgccattatttagtggatttggacaccatgagagaaacaccccgggagccaaaatattcatccaataaagaagaatggatcagcttggcctatagaccattccttgatgcttctagatcttcaaagctgctgcgggcattctatgtccccttcctgtcagatcagtatacagtgtacgtaaactacaccatcctcaaaccccggaaagcaaagcaaatcaggaagaaaagtggaggttag

SEQ ID NO: 6 - protein, 618 aa, Homo sapiens
MASRGARQRLKGSGASSGDTAPAADKLRELLGSREAGGAEHRTELSGNKAGQVWAPEGSTAFKCLLSARLCAALLSNISDCDETFNYWEPTHYLIYGEGFQTWEYSPAYAIRSYAYLLLHAWPAAFHARILQTNKILVFYFLRCLLAFVSCICELYFYKAVCKKFGLHVSRMMLAFLVLSTGMFCSSSAFLPSSFCMYTTLIAMTGWYMDKTSIAVLGVAAGAILGWPFSAALGLPIAFDLLVMKHRWKSFFHWSLMALILFLVPVVVIDSYYYGKLVIAPLNIVLYNIFTPHGPDLYGTEPWYFYLINGFLNFNVAFALALLVLPLTSLMEYLLQRFHVQNLGHPYWLTLAPMYIWFIIFFIQPHKEERFLFPVYPLICLCGAVALSALQHSFLYFQKCYHFVFQRYRLEHYTVTSNWLALGTVFLFGLLSFSRSVALFRGYHGPLDLYPEFYRIATDPTIHTVPEGRPVNVCVGKEWYRFPSSFLLPDNWQLQFIPSEFRGQLPKPFAEGPLATRIVPTDMNDQNLEEPSRYIDISKCHYLVDLDTMRETPREPKYSSNKEEWISLAYRPFLDASRSSKLLRAFYVPFLSDQYTVYVNYTILKPRKAKQIRKKSGG

SEQ ID NO: 7 - DNA, 1422 bp, Homo sapiens
  CDS (1)..(1422)
atggcgcagctggaaggttactatttctcggccgccttgagctgtacctttttagtatcctgcctcctcttctccgccttcagccgggcgttgcgagagccctacatggacgagatcttccacctgcctcaggcgcagcgctactgtgagggccatttctccctttcccagtgggatcccatgattactacattacctggcttgtacctggtgtcaattggagtgatcaaacctgccatttggatctttggatggtctgaacatgttgtctgctccattgggatgctcagatttgttaatcttctcttcagtgttggcaacttctatttactatatttgcttttctgcaaggtacaacccagaaacaaggctgcctcaagtatccagagagtcttgtcaacattaacactagcagtatttccaacactttatttttttaacttcctttattatacagaagcaggatctatgttttttactctttttgcgtatttgatgtgtctttatggaaatcataaaacttcagccttccttggattttgtggcttcatgtttcggcaaacaaatatcatctgggctgtcttctgtgcaggaaatgtcattgcacaaaagttaacggaggcttggaaaactgagctacaaaagaaggaagacagacttccacctattaaaggaccatttgcagaattcagaaaaattcttcagtttcttttggcttattccatgtcctttaaaaacttgagtatgcttttgcttctgacttggccctacatccttctgggatttctgttttgtgcttttgtagtagttaatggtggaattgttattggcgatcggagtagtcatgaagcctgtcttcattttcctcaactattctactttttttcatttactctctttttttcctttcctcatctcctgtctcctagcaaaattaagacttttctttccttagtttggaaacgtagaattctgttttttgtggttaccttagtctctgtgtttttagtttggaaattcacttatgctcataaatacttgctagcagacaatagacattatactttctatgtgtggaaaagagtttttcaaagatatgaaactgtaaaatatttgttagttccagcctatatatttgctggttggagtatagctgactcattgaaatcaaagtcaattttttggaatttaatgtttttcatatgcttgttcactgttatagttcctcagaaactgctggaatttcgttacttcattttaccttatgtcatttataggcttaacatacctctgcctcccacatccagactcatttgtgaactgagctgctatgcagttgttaatttcataacttttttcatctttctgaacaagacttttcagtggccaaatagtcaggacattcaaaggtttatgtggtaa

SEQ ID NO: 8 - protein, 473 aa, Homo sapiens
MAQLEGYYFSAALSCTFLVSCLLFSAFSRALREPYMDEIFHLPQAQRYCEGHFSLSQWDPMITTLPGLYLVSIGVIKPAIWIFGWSEHVVCSIGMLRFVNLLFSVGNFYLLYLLFCKVQPRNKAASSIQRVLSTLTLAVFPTLYFFNFLYYTEAGSMFFTLFAYLMCLYGNHKTSAFLGFCGFMFRQTNIIWAVFCAGNVIAQKLTEAWKTELQKKEDRLPPIKGPFAEFRKILQFLLAYSMSFKNLSMLLLLTWPYILLGFLFCAFVVVNGGIVIGDRSSHEACLHFPQLFYFFSFTLFFSFPHLLSPSKIKTFLSLVWKRRILFFVVTLVSVFLVWKFTYAHKYLLADNRHYTFYVWKRVFQRYETVKYLLVPAYIFAGWSIADSLKSKSIFWNLMFFICLFTVIVPQKLLEFRYFILPYVIYRLNIPLPPTSRLICELSCYAVVNFITFFIFLNKTFQWPNSQDIQRFMW

SEQ ID NO: 9 - DNA, 1467 bp, Homo sapiens
  CDS (1)..(1467)
atggctggaaaggggtcatcaggcaggcggcccctgctgctggggctgctggtggccgtagccactgtccacctggtcatctgtccctacaccaaagtggaggagagcttcaacctgcaggccacacatgacctgctctaccactggcaagacctggagcagtacgaccatcttgagttccccggagtcgtccccaggacgttcctcgggccagtggtgatcgcagtgttctccagccccgcggtttacgtgctttcgctgttagaaatgtccaagttttactctcagctaatagttagaggagtgcttggactcggcgtgatttttggactctggacgttacaaaaggaagtgagacggcacttcggggccatggtggccaccatgttctgctgggtgacggccatgcagttccacctgatgttctactgcacgcggacactgcccaatgtgctggccctgcctgtagtcctgctggccctcgcggcctggctgcggcacgagtgggcccgcttcatctggctgtcagccttcgccatcatcgtgttcagggtggagctgtgcctgttcctgggcctcctgctgctgctggccttgggcaaccgaaaggtttctgtagtcagagcccttcgccacgccgtcccggcagggatcctctgtttaggactgacggttgctgtggactcttatttttggcggcagctcacttggccggaaggaaaggtgctttggtacaacactgtcctgaacaaaagctccaactgggggacctccccgctgctgtggtacttctactcagccctgccccgcggcctgggctgcagcctgctcttcatccccctgggcttggtagacagaaggacgcacgcgccgacggtgctggcactgggcttcatggcactctactccctcctgccacacaaggagctacgcttcatcatctatgccttccccatgctcaacatcacggctgccagaggctgctcctacctgctgaataactataaaaagtcttggctgtacaaagcggggtctctgcttgtgatcggacacctcgtggtgaatgccgcctactcagccacggccctgtatgtgtcccatttcaactacccaggtggcgtcgcaatgcagaggctgcaccagctggtgcccccccagacagacgtccttctgcacattgacgtggcagccgcccagacaggtgtgtctcggtttctccaagtcaacagcgcctggaggtacgacaagagggaggatgtgcagccggggacaggcatgctggcatacacacacatcctcatggaggcggcccctgggctcctggccctctacagggacacacaccgggtcctggccagcgtcgtggggaccacaggtgtgagtctgaacctgacccaactgccccccttcaacgtccacctgcagacaaagctggtgcttctggagaggctcccccggccgtcctga

SEQ ID NO: 10 - protein, 488 aa, Homo sapiens
MAGKGSSGRRPLLLGLLVAVATVHLVICPYTKVEESFNLQATHDLLYHWQDLEQYDHLEFPGVVPRTFLGPVVIAVFSSPAVYVLSLLEMSKFYSQLIVRGVLGLGVIFGLWTLQKEVRRHFGAMVATMFCWVTAMQFHLMFYCTRTLPNVLALPVVLLALAAWLRHEWARFIWLSAFAIIVFRVELCLFLGLLLLLALGNRKVSVVRALRHAVPAGILCLGLTVAVDSYFWRQLTWPEGKVLWYNTVLNKSSNWGTSPLLWYFYSALPRGLGCSLLFIPLGLVDRRTHAPTVLALGFMALYSLLPHKELRFIIYAFPMLNITAARGCSYLLNNYKKSWLYKAGSLLVIGHLVVNAAYSATALYVSHFNYPGGVAMQRLHQLVPPQTDVLLHIDVAAAQTGVSRFLQVNSAWRYDKREDVQPGTGMLAYTHILMEAAPGLLALYRDTHRVLASVVGTTGVSLNLTQLPPFNVHLQTKLVLLERLPRPS

(19) United States
(12) Patent Application Publication
(10) Pub. No.: US 2006/0257387 A1
(43) Pub. Date: Nov. 16, 2006

(54) Sugar chain synthase gene

(75) Inventors: Kenichi Nakayama, Ibaraki (JP); Tomoko Ishii, Ibaraki (JP); Yoshihumi Jigami, Ibaraki (JP)

Correspondence Address: MORGAN LEWIS & BOCKIUS LLP, 1111 PENNSYLVANIA AVENUE NW, WASHINGTON, DC, 20004, US

(73) Assignee: National Institute Of Advanced Industrial Science And Technology, Chiyoda-Ku, Tokyo (JP)

(21) Appl. No.: 10/523,363

(22) PCT Filed: Aug. 6, 2003
(86) PCT No.: PCT/JP03/10025

§ 371 Date: Aug. 31, 2005

(30) Foreign Application Priority Data
Aug. 7, 2002 (JP) 2002-230196

(51) Int. Cl. C12Q 1/68; C07H 21/04; C12P 21/06; A61K 38/47; C12N 9/10; C12N 1/18

(52) U.S. Cl. 424/94.61; 435/69.1; 435/193; 435/320.1; 435/254.2; 435/6; 536/23.2

(57) ABSTRACT

The present invention provides a method effective for diagnosis or treatment of congenital disorders of glycosylation syndrome (CDGS) by clarifying the gene of the N-linked sugar chain synthase in human endoplasmic reticulum. In the present invention, a gene of an enzyme catalyzing human N-linked sugar chain synthesis is found based on, as indicators, whether it is homologous with the gene of the enzyme catalyzing N-linked sugar chain synthesis in yeast endoplasmic reticulum and compliments the function of the gene for a deletion yeast strain of the gene.

### TECHNICAL FIELD

The present invention relates to a human gene for synthesizing a human-derived N-linked sugar chain; an agent for diagnosing or treating congenital disorders of glycosylation syndrome (CDGS) by using the gene; a recombinant vector and a transformant which are integrated with the gene; a process for producing an enzyme catalyzing a human N-linked sugar chain synthesis by using the transformant; or a method for synthesizing a human N-linked sugar chain by using the enzyme or the transformant.

### BACKGROUND ART

In order to identify a causative gene of congenital disorders of glycosylation syndrome (CDGS), it is required that genes relating to sugar chain synthesis should comprehensively be cloned. Particularly, genes in the synthetic processes in human endoplasmic reticulum which relate to the essential synthesis of a human N-linked sugar chain are particularly important.

Actually, some causative genes of congenital disorders of glycosylation syndrome have been investigated. However, it is known that the syndrome is mostly caused by genes relating to the synthesis of an N-linked sugar chain in endoplasmic reticulum. The synthetic pathway of an N-linked sugar chain in endoplasmic reticulum is conserved in yeast up to humans. Most of the genes relating to the synthesis have been isolated from yeast.

Although it is considered that almost all sequences of human genes exist on database, most of the genes have not yet been isolated because the functions are unknown. Thus, isolation of these genes is an important problem for further detailed diagnosis and treatment of CDGS.

On the other hand, the enzymes for the fundamental biosynthesis in endoplastic reticulum for the synthesis of an N-linked sugar chain are essential for the synthesis in vitro at a large scale. Therefore, the isolation of these genes is very important for the supply of the enzymes as an application to sugar chain engineering.

According to the present invention, the human gene relating to the N-linked sugar chain synthesis in endoplasmic reticulum is clarified, congenital disorders of glycosylation syndrome is diagnosed and treated by using it, and a method for synthesizing the enzyme at a large scale is provided as an application to sugar chain engineering.

### DISCLOSURE OF THE INVENTION

The inventors made intensive studies in order to solve the problems, and found a human gene which is highly homologous with an enzyme catalyzing N-linked sugar chain synthesis in yeast endoplasmic reticulum and then cloned it. Surprisingly, the cloned human gene complimented the function of the gene for a deletion strain of the gene in yeast endoplastic reticulum. Thus, the inventors were convinced that the human gene would be a gene of an N-linked sugar chain synthase in human endoplasmic reticulum. Thus, the present invention has been achieved.

The present invention relates to the followings.
(1) A human gene for synthesizing an enzyme catalyzing human N-linked sugar chain synthesis, which is homologous with a gene of an enzyme catalyzing N-linked sugar chain synthesis in yeast endoplasmic reticulum, and is capable of complimenting the function of said gene for a deletion yeast strain of said gene.
(2) The human gene according to the above-described (1), wherein the enzyme catalyzing human N-linked sugar chain synthesis is a glycosyltransferase.
(3) A gene which encodes the amino acid sequence represented by SEQ ID NO:2, 4, 6, 8 or 10 or a protein which comprises an amino acid sequence in which one or more amino acids in the amino acid sequence represented by SEQ ID NO:2, 4, 6, 8 or 10 are deleted, substituted or added.
(4) A gene which comprises the nucleotide sequence represented by SEQ ID NO:1, 3, 5, 7 or 9.
(5) An agent for diagnosing or treating human congenital disorders of glycosylation syndrome, which comprises using the gene encoding the amino acid sequence according to the above-described (3) or the gene represented by SEQ ID NO: 1, 3, 5, 7 or 9.
(6) A recombinant vector which is integrated with the gene according to any one of the above-described (1) to (3).
(7) A transformant which is transformed by the recombinant vector according to the above-described (6).
(8) A process for producing an enzyme catalyzing human N-linked sugar chain synthesis, which comprises culturing the transformant according to the above-described
(7) in a culture, and collecting the enzyme catalyzing human N-linked sugar chain synthesis from the culture.
(9) A method for synthesizing a human N-linked sugar chain, which comprises using the enzyme according to the above-described (8).

### BRIEF DESCRIPTION OF THE DRAWINGS

FIG. 1 shows results of electrophoresis of a transformant sample, a sample of JY746 strain (wild type strain) and a sample of gmd3 strain.

FIG. 2 shows results of electrophoresis of a transformant sample, a sample of W303-1A strain (wild type strain) and a sample of alg8 strain.

FIG. 3 shows results of electrophoresis of a transformant sample, a sample of W303-1A strain (wild type strain) and a sample of alg9 strain.

FIG. 4 shows results of electrophoresis of a transformant sample, a sample of W303-1A strain (wild type strain) and a sample of alg10 strain.

FIG. 5 shows results of electrophoresis of a transformant sample, a sample of W303-1A strain (wild type strain) and a sample of alg12 strain.

### BEST MODE FOR CARRYING OUT THE INVENTION

In the present invention, genes for use in cloning gene of an enzyme catalyzing human N-linked sugar chain synthesis are genes of an enzyme group relating to N-linked sugar chain synthesis in yeast endoplasmic reticulum, such as genes of ALG11 gene, ALG8 gene, ALG9 gene, ALG10 gene and ALG12 gene. Examples include alg11 gene of Schizosaccharomyces pombe, ALG8 gene, ALG9 gene, ALG10 gene and ALG12 gene of Saccharomyces cerevisiae, and the like.

The alg11 gene of Schizosaccharomyces pombe is a gene encoding glycolipid α-mannosyltransferase (EC 2.4.1.131) in the N-linked sugar chain synthesis system.

The ALG8 gene of Saccharomyces cerevisiae is a gene encoding glycolipid α-glycosyltransferases (EC 2.4.1._) in the N-linked sugar chain synthesis system.

The ALG9 gene of Saccharomyces cerevisiae is a gene encoding glycolipid α-mannosyltransferase (EC 2.4.1.130) in the N-linked sugar chain synthesis system.

The ALG10 gene of Saccharomyces cerevisiae is a gene encoding glycolipid α-glucosyltransferases (EC 2.4.1._) in the N-linked sugar chain synthesis system.

The ALG12 gene of Saccharomyces cerevisiae is a gene encoding glycolipid α-mannosyltransferase (EC 2.4.1.130) in the N-linked sugar chain synthesis system.

A human gene which is homologous with these yeast genes and is capable of complimenting the function for a deletion or mutation yeast strain of these genes is a gene of an enzyme in the N-linked sugar chain synthesis system in human endoplasmic reticulum.

In order to obtain the gene of the enzyme in the N-linked sugar chain synthesis system in human endoplasmic reticulum in the present invention, a human gene which is homologous with a gene of an enzyme in the N-linked sugar chain synthesis system in yeast endoplasmic reticulum is cloned. In this cloning, human cloned DNA which is homologous with the gene of the enzyme in the N-linked sugar chain synthesis system in yeast endoplasmic reticulum can be obtained, for example, by preparing synthetic primers based on the nucleotide sequence of the gene of the enzyme in the N-linked sugar chain synthesis system in yeast and carrying out PCR using a human cDNA library.

Then, the human gene which is homologous with the gene of the enzyme in the N-linked sugar chain synthesis system in yeast endoplasmic reticulum is ligated to a vector which can be expressed in yeast, such as pREP1, YEp51, YEp352GAP, pSH19, and pYO325, to transform yeast in which the gene of the enzyme in the N-linked sugar chain synthesis system is deleted or mutated with the recombinant expression vector. When the transformed yeast recovers the function lost by the deletion or mutation of the gene of the enzyme, the human gene is considered to be the gene of the enzyme in the N-linked sugar chain synthesis system in human endoplasmic reticulum. Then, a large number of the recombinant vectors are collected by PCR amplification or culturing of the transformant, and the gene of the enzyme in the N-linked sugar chain synthesis system in human endoplasmic reticulum can be obtained by known methods in the field, such as cleavage of the vector with restriction enzymes.

In this point, more specifically, for example, the alg11 gene of Schizosaccharomyces pombe is a gene encoding glycolipid α-mannosyltransferase (EC2.4.1.131) in the N-linked sugar chain synthesis system, and the gmd3 strain of Schizosaccharomyces pombe in which this gene is mutated is temperature-sensitive and is deficient in sugar chain addition, so that it produces an acidic phosphatase having a molecular weight smaller than that of the wild type strain because sugar chain addition of the acidic phosphatase which is a glycoprotein is deficient. In this connection, a human gene (for example, FLJ21803) which is highly homologous with the alg11 gene is obtained by preparing primers based on the sequence of the alg11 gene and carrying out amplification by PCR using a human cDNA library. Using the gene, the gmd3 strain is transformed to thereby examine the temperature sensitivity and the molecular weight of the acidic phosphatase. When the transformant is negative with the temperature sensitivity and the molecular weight of the acidic phosphatase is returned to the same level as that of the wild type strain, the human gene is the gene of the enzyme in the N-linked sugar chain synthesis in human endoplasmic reticulum and can be produced in a large amount according to the usual method.

The yeast strain in which the gene of the enzyme in the N-linked sugar chain synthesis system is deleted or mutated includes Schizosaccharomyces pombe gmd3 strain in which the alg11 gene is mutated, Saccharomyces cerevisiae alg8 strain in which the ALG8 gene is mutated, and the like. The yeast strain can be obtained by mutating yeast according to a mutagenic method, such as radiation and ultraviolet irradiation, and screening a yeast strain in which the gene of the enzyme in the N-linked sugar chain synthesis is deleted or mutated by using decrease of the molecular weight of the glycoprotein or the temperature sensitivity as an indicator.

CDGS is an autosomal recessive genetic disease and causes various disorders such as cerebellar hypoplasia, liver disorders and peripheral nerve disorders. Among these, it is considered that type I CDGS is caused by the deficiency of the enzyme due to the deletion or mutation of the gene of the enzyme in the N-linked sugar chain synthesis. The gene identified as the gene of the enzyme in the human N-linked sugar chain synthesis system for the first time in the present invention is a useful diagnostic agent of CDGS. Whether or not a patient suffers from CDGS can be diagnosed by comparing the nucleotide sequence of glycolipid α-mannosyltransferase (EC 2.4.1.131) represented by SEQ ID NO:1 with the nucleotide sequence of the corresponding gene of the enzyme of the patient to detect abnormality of the gene.

When the diagnosis is carried out by using the gene of the enzyme in the human N-linked sugar chain synthesis system in the present invention, the gene of the enzyme of the patient as a comparative subject can be obtained by collecting the subject gene from patient blood using the gene of the enzyme of the present invention as probe and amplifying it appropriately by PCR.

The gene of the enzyme in the human N-linked sugar chain synthesis system in the present invention is also useful for gene therapy. For the therapy, the gene of the enzyme in the human N-linked sugar chain synthesis system in the present invention is inserted into a vector for gene therapy, such as adenovirus, retrovirus or Sendai virus, to prepare viral particles containing the gene using helper cells or the like, and the viral particles are inoculated into human bodies to introduce the gene of the enzyme.

In the present invention, the gene of the enzyme in the human N-linked sugar chain synthesis system is inserted into a vector, such as plasmid pBR322, pUC18, pUC19, pET-3, YEp51 or YEp352GP, to transform a host, such as bacterium or yeast, with the vector. The resulting transformant is cultured in a culture to prepare the enzyme in the human N-linked sugar chain synthesis system corresponding to the gene in a large amount. The vector for use in the method for producing the enzyme of the present invention includes pBR322, pUC18, pET-3 and the like when the host is Escherichia coli, and YEp13, YCp50, YEp51, YEp352GAP, pSH19, pREP1 and the like when the host is yeast. Furthermore, a promoter is linked to the upstream thereof so as to express the gene. The promoter used in the present invention may be any promoter, so long as it is a suitable promoter corresponding to the host used for expressing the gene. The host includes Escherichia coli (BL21, BL21(DE3), etc.), yeast (Saccharomyces cerevisiae, Pichia pastoris, Schizosaccharomiyces pombe, etc.), and the like.

The enzyme in the human N-linked sugar chain synthesis system obtained by the above production method is useful as a therapeutic agent of CDGS, and a human N-linked sugar chain can be synthesized in vitro by using the enzyme. For example, in the case of α-mannosyltransferase encoded by the human ALG 11 homologous gene, Man5GlcNAc2-pp-Dol can be synthesized by using Man4GlcNAc2-pp-Dol and GDP-mannose as substrates.

Examples of the present invention are shown below; however, particularly, the present invention is not limited thereto.

### EXAMPLE 1

### Cloning of ALG11 Human Homolog FLJ21803

The gene was cloned by PCR using a human cDNA library. As the human cDNA library, QUICK.Clone cDNA manufactured by CLONTECH was used. As primers, primers containing an NdeI site at the N-terminal and an SmaI site at the C-terminal were prepared in advance, on the basis of sequences registered on the database so as to easily cleave a part encoding the protein with restriction enzymes. Sequences of respective primers are shown below.

5′-TCCCCCGGGT TACTTAAATA ACTTTTCCAC AGATGATAGG
AA-3′
5′-GGGAATTCCA TATGGCGGCC GGCGAAAGGA GCTG-3′

PCR conditions are as follows:

| | |
|---|---|
| First stage: | 94° C., 15 seconds |
| Second stage: | 49° C., 30 seconds |
| Third stage: | 72° C., 3 minutes |
| 30 Cycles | |

A DNA amplification fragment of about 1.5 kbp obtained under the conditions was inserted into a pCR2.1TOPO vector by using a TA cloning kit. The nucleotide sequence of the cloned gene was confirmed by a sequence kit using the dideoxy method. The gene had the nucleotide sequence represented by SEQ ID NO: 1. Furthermore, SEQ ID NO: 1 also shows the amino acid sequence corresponding to the nucleotide sequence of the gene. Additionally, the amino acid sequence of a protein corresponding to the gene is represented by SEQ ID NO:2.

Transformation:

The FLJ21803 gene inserted into the pCR2.1TOPO vector was cleaved by NdeI-SmaI, and inserted into the NdeI-SmaI site of a vector for fission yeast multicopy expression, pREP1, having a multicloning site between the promoter nmt1 of fission yeast and the terminator thereof to thereby construct 21803/pREP1. The expression vector was transformed into Schizosaccharomyces pombe gmd3 mutant strain of fission yeast.

Function of gmd3 Mutant Strain:

The temperature sensitivity of the resulting transformant was examined, which was an indicator of the presence or absence of N-linked sugar chain synthesis. The transformant and Schizosaccharomyces pombe JY746 strain (wild type strain) and the gmd3 strain which were controls were cultured in an MM-leu medium having the following composition at 37° C. for 3 days to examine the temperature sensitivity.

As a result, it was confirmed that the transformant and the wild type strain could grow even at 37° C. On the other hand, the gmd3 strain could not grow at 37° C.

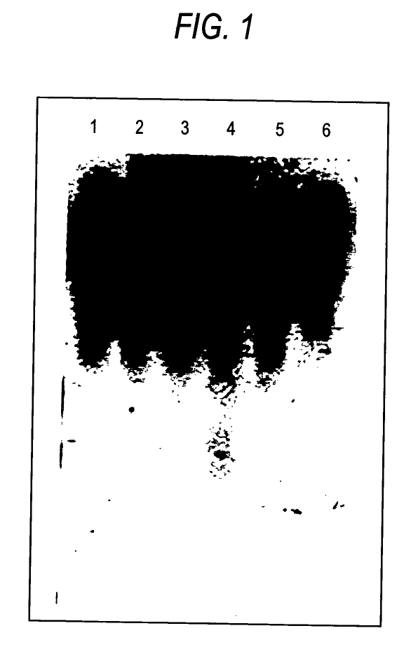

Separately, the transformant which could grow at 37° C. was collected, and was grown in a low phosphoric acid medium and disrupted with glass beads. The resulting product was used as a sample for electrophoresis on acrylamide gel. In the same manner, electrophoresis was carried out by using a sample obtained from the gmd3 strain in which the ALG11 gene of Saccharomyces cerevisiae was transformed, a sample of the gmd3 strain, and a sample of the JY746 strain (wild type strain). The results are collectively shown in FIG. 1.

In the drawing, lanes 1 to 3 in the drawing show samples of the transformant of Schizosaccharomyces pombe, lane 4 shows a sample of the gmd3 strain obtained by separate culture, lane 5 shows a sample obtained from the gmd3 strain in which the ALG11 gene of Saccharomyces cerevisiae was transformed, and lane 6 shows a sample obtained from the JY746 strain (wild type strain).

In both of the transformant samples and the sample of the JY746 strain (wild type strain), bands corresponding to an acidic phosphatase having a large molecular weight to which sugar chains were completely added were observed, whereas, in the sample of the gmd3 strain, only a band of an acidic phosphatase having a molecular weight smaller than that of the bands, to which sugar chains were incompletely added, was observed.

Accordingly, these results clearly show that the human gene FLJ21803 compliments the function in fission yeast.

### EXAMPLE 2

### Cloning of ALG8 Human Homolog MGC2840

Using a human cDNA library, the gene was cloned by PCR. As the human cDNA library, QUICK-Clone cDNA manufactured by CLONTECH was used. The primers were prepared on the basis of sequences registered on the database. Sequences of the respective primers are shown below.

5′-GGAATTCCAT ATGGCGGCGC TCACAATTG CCACGGGTAC
TGGC-3′
5′-TCCCCCGGGT CATTGTTTCT TTGTCTTGC CAATAGCAGA G-3′

PCR conditions are as follows:

| | |
|---|---|
| First stage: | 94° C., 30 seconds |
| Second stage: | 50° C., 30 seconds |
| Third stage: | 72° C., 2 minutes |
| 30 Cycles | |

A DNA amplification fragment of about 1.5 kbp obtained under the conditions was inserted into a pCR2.1TOPO vector by using a TA cloning kit. The nucleotide sequence of the cloned gene was confirmed by a sequence kit using the dideoxy method. The gene had the nucleotide sequence represented by SEQ ID NO:3. Furthermore, SEQ ID NO:4 shows the amino acid sequence corresponding to the gene.

Transformation:

The MGC2840 gene inserted into the pCR2.1TOPO vector was cleaved by EcoRI-NaeI, and then inserted into the EcoRI-PvuII site of a vector for expression, YEp352GAO, having a part from the EcoRI region to the SalI region in the multicloning site of pUC18 between a promoter GAPDH in the yeast glycolytic pathway and the terminator thereof. These expression vectors were transformed into Saccharomyces cerevisiae alg8 wbp1 mutant strain of budding yeast.

Recovery of Function of alg8 wbp1 Mutant Strain:

The temperature sensitivity of the resulting transformant was examined, which was an indicator of the presence or absence of N-linked sugar chain synthesis. The transformant and Saccharomyces cerevisiae W303-1A strain (wild type strain) and the alg8 wbp1 mutant strain which were controls were cultured in an SD-ura medium having the following composition at 30° C. for 5 days to examine the temperature sensitivity.

As a result, it was confirmed that the transformant and the wild type strain could grow even at 30° C. On the other hand, the alg8 wbp1 mutant strain could not grow at 30° C.

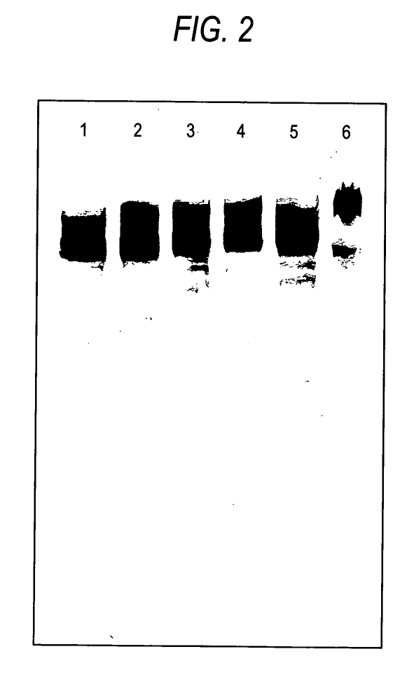

Separately, the transformant which could grow at 30° C. was collected, and was grown in a complete medium and disrupted with glass beads. The resulting product was used as a sample for electrophoresis on acrylamide gel. In the same manner, a sample of the alg8 wbp1 strain and a sample of the W303-1A strain (wild type strain) were subjected to electrophoresis. The results are collectively shown in FIG. 2.

In the drawing, lanes 1 to 4 show samples obtained from the transformant of Saccharomyces cerevisiae, lane 5 shows a sample of the alg8 wbp1 strain obtained by separate culture, and lane 6 shows a sample obtained from the W303-1A strain (wild type strain). In both of the transformant sample and the sample of the W303-1A strain (wild type strain), bands corresponding to a carboxypeptidase Y having a large molecular weight to which sugar chains were completed added were observed, whereas, in the sample of the alg8 wbp1, only a band of a carboxypeptidase Y having a molecular weight smaller than that of the bands, to which sugar chains were incompletely added, was observed.

Accordingly, these results clearly show that the human gene MGC2840 compliments the function in budding yeast.

### EXAMPLE 3

### Cloning of ALG9 Human Homolog FLJ21845

Using a human cDNA library, the gene was cloned by PCR. As the human cDNA library, QUICK-Clone cDNA manufactured by CLONTECH was used. As the primers, primers were prepared on the basis of sequences registered on the database. Sequences of respective primers are shown below.

5′-AACGTTAACA TGGCTAGTCG AGGGGCTCGG CAGCGCCTGA AGGGCAGC-3′
5′-AACGTTAACC TAACCTCCAC TTTTCTTCCT GATTTGCTTT GCTTTCCG-3′

PCR conditions are as follows:

| | |
|---|---|
| First stage: | 94° C., 30 seconds |
| Second stage: | 50° C., 30 seconds |
| Third stage: | 72° C., 3 minutes |
| 30 Cycles | |

A DNA amplification fragment of about 2 kbp obtained under the conditions was inserted into a pCR2.1TOPO vector by using a TA cloning kit. The nucleotide sequence of the cloned gene was confirmed by a sequence kit using the dideoxy method. The gene had the nucleotide sequence represented by SEQ ID NO:5. Furthermore, SEQ ID NO:6 shows the amino acid sequence corresponding to the gene.

Transformation:

The FLJ21845 gene inserted into the pCR2.1TOPO vector was cleaved by EcoRI-DraI and then inserted into the EcoRI-PvuII site of a vector for expression, YEp352GAP, having a part from the EcoRI region to the SalI region in the multicloning site of pUC18 between a promoter GAPDH in the yeast glycolytic pathway and the terminator thereof. These expression vectors were transformed into Saccharomyces cerevisiae alg9 wbp1 mutant strain of budding yeast.

Recovery of Function of alg9 wbp1 Mutant Strain

The temperature sensitivity of the resulting transformant was examined, which was an indicator of the presence or absence of N-linked sugar chain synthesis. The transformant and Saccharomyces cerevisiae W303-1A strain (wild type strain) and the alg9 wbp1 mutant strain which were controls were cultured in an SD-ura medium having the following composition at 30° C. for 5 days to examine the temperature sensitivity. As a result, it was confirmed that the transformant and the wild type strain could grow even at 30° C. On the other hand, the alg9 wbp1 mutant strain could not grow at 30° C.

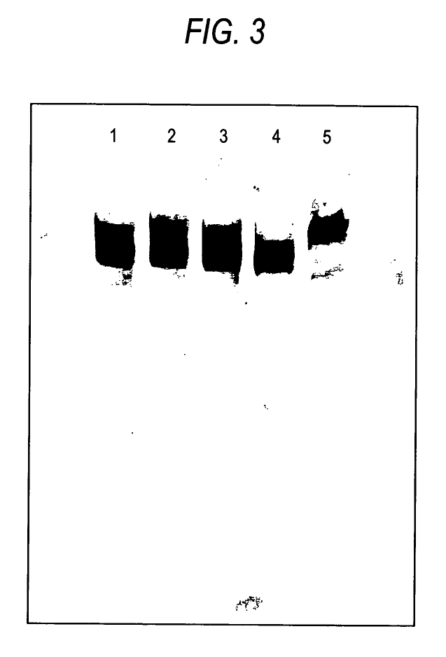

Separately, the transformant which could grow at 30° C. was collected, and was grown in a complete medium and disrupted with glass beads. The resulting product was used as a sample for electrophoresis on acrylamide gel. In the same manner, a sample of the alg9 wbp1 strain and a sample of the W303-1A strain (wild type strain) were subjected to electrophoresis. The results are collectively shown in FIG. 3.

In the drawings, lanes 1 to 3 show samples obtained from the transformant of Saccharomyces cerevisiae, lane 4 shows the sample of the alg9 wbp1 strain obtained by separate culture, lane 5 shows a sample obtained from the W303-1A strain (wild type strain).

In both of the transformant sample and the sample of the W303-1A strain (wild type strain), bands corresponding to a carboxypeptidase Y having a large molecular weight to which sugar chains were completely added were observed, whereas, in the sample of the alg9 wbp1, only a band of a carboxypeptidase Y having a molecular weight smaller than that of the bands, to which sugar chains were not added, was observed.

Accordingly, these results clearly show that the human gene FLJ21845 compliments the function in budding yeast.

### EXAMPLE 4

### Cloning of ALG10 Human Homolog XM_{—}050190

Using a human cDNA, the gene was cloned by PCR. As the cDNA, human stomach cDNA was used. As the primers, primers were prepared on the basis of sequences registered on the database. Sequences of respective primers are shown below.

5′-AAAAGGCCTA TGGCGCAGCT GGAAGGTTAC TATTTCTCGG CCGCCTTG-3′
5′-TTTTCCGGAT TACCACATAA ACCTTTGAAT GTCCTGACTA TTTGGCCA CT-3′

PCR conditions are as follows:

| | |
|---|---|
| First stage: | 94° C., 30 seconds |
| Second stage: | 50° C., 30 seconds |
| Third stage: | 72° C., 2 minutes |
| 30 Cycles | |

A DNA amplification fragment of about 1.5 kbp obtained under the conditions was inserted into a pCR2.1TOPO vector by using a TA cloning kit. The nucleotide sequence of the cloned gene was confirmed by a sequence kit using the dideoxy method. The gene had the nucleotide sequence represented by SEQ ID NO:7. Furthermore, SEQ ID NO:8 shows the amino acid sequence corresponding to the gene.

Transformation:

The M_{—}050190 gene inserted into the pCR2.1TOPO vector was cleaved by EcoRI-KpnI and then inserted into the EcoRI-KpnI site of a vector for expression, YEp352GAO, having a part from the EcoRI region to the SalI region in the multicloning site of pUC18 between a promoter GAPDH in the yeast glycolytic pathway and the terminator thereof. These expression vectors were transformed into Saccharomyces cerevisiae alg10 wbp1 mutant strain of budding yeast.

Recovery of Function of alg10 wbp1 Mutant Strain:

The temperature sensitivity of the resulting transformant was examined, which was an indicator of the presence or absence of N-linked sugar chain synthesis. The transformant and Saccharomyces cerevisiae W303-1A strain (wild type strain) and the alg10 wbp1 mutant strain which were controls were cultured in an SD-ura medium having the following composition at 30° C. for 5 days to examine the temperature sensitivity.

As a result, it was confirmed that the transformant and the wild type strain could grow even at 30° C. On the other hand, the alg10 wbp1 mutant strain could not grow at 30° C.

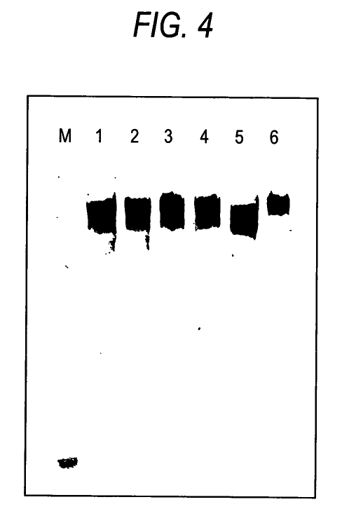

Separately, the transformant which could grow at 30° C. was collected, and was grown in a complete medium and disrupted with glass beads. The resulting product was used as a sample for electrophoresis on acrylamide gel. In the same manner, a sample of the alg10 wbp1 strain and a sample of the W303-1A strain (wild type strain) were subjected to electrophoresis. The results are collectively shown in FIG. 4.

In the drawing, lanes 1 to 4 show samples obtained from the transformant of Saccharomyces cerevisiae, lane 5 shows the sample of the alg10 wbp1 strain obtained by separate culture, and lane 6 shows a sample obtained from the W303-1A strain (wild type strain).

In both of the transformant sample and the sample of the W303-1A strain (wild type strain), bands corresponding to a carboxypeptidase Y having a large molecular weight to which sugar chains were completely added were observed, whereas, in the alg10 wbp1 sample, a band of a carboxypeptidase Y having a molecular weight smaller than that of the bands, to which sugar chains were not added, was observed.

Accordingly, these results clearly show that the human gene XM_{—}050190 compliments the function in budding yeast.

### EXAMPLE 5

### Cloning of ALG12 Human Homolog MGC3136

Using a human cDNA library, the gene was cloned by PCR. As the human cDNA library, human tissue cDNA was used. As the primers, primers were prepared on the basis of sequences registered on the database. Sequences of respective primers are shown below.

5′-CGGAATTCAT GGCTGGAAAG GGGTCATCAG GCAGGCGG-3′
5′-CGGAATTCTC AGGACGGCCG GGGGAGCCTC TCCAGAAGC-3′

PCR conditions are as follows:

| | |
|---|---|
| First stage: | 94° C., 30 seconds |
| Second stage: | 50° C., 30 seconds |
| Third stage: | 72° C., 3 minutes |
| 30 Cycles | |

A DNA amplification fragment of about 1.5 kbp obtained under the conditions was inserted into a pCR2.1TOPO vector by using a TA cloning kit. The nucleotide sequence of the cloned gene was confirmed by a sequence kit using the dideoxy method. The gene had the nucleotide sequence represented by SEQ ID NO:9. Furthermore, SEQ ID NO: 10 shows the amino acid sequence of a protein corresponding to the gene.

Transformation:

The MGC3136 gene inserted into the pCR2.1TOPO vector was cleaved by EcoRI and then inserted into the EcoRI site of a expression for expression, YEp352GAO, having a part from the EcoRI region to the SalI region in the multicloning site of pUC18 between a promoter GAPDH in the yeast glycolytic pathway and the terminator thereof. These expression vectors were transformed into Saccharomyces cerevisiae alg12 mutant strain of budding yeast.

Recovery of Function of alg12 Mutant Strain:

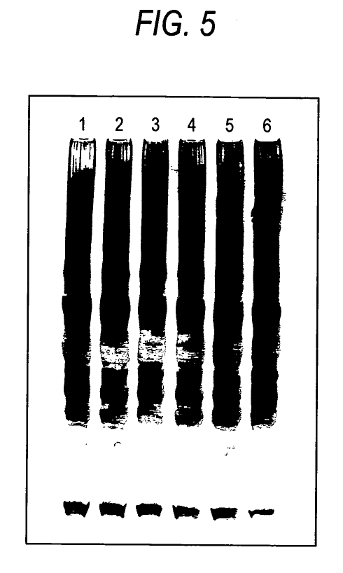

The resulting transformant was collected, grown in a complete medium and disrupted with glass beads. The resulting product was used as a sample for electrophoresis using acrylamide gel. In the same manner, a sample of the alg12 strain and a sample of the W303-1A strain (wild type strain) were also subjected to electrophoresis. The results are collectively shown in FIG. 5.

In the drawing, lanes 1 to 4 show samples obtained from the transformant of Saccharomyces cerevisiae, lane 5 shows the sample of the alg12 strain obtained by separate culture, and lane 6 shows a sample obtained from the W303-1A strain (wild type strain).

In the transformant sample and the sample of the W303-1A strain (wild type strain), bands corresponding to a carboxypeptidase Y having a large molecular weight to which sugar chains were completely added were observed, whereas, in the sample of the alg12 strain, only a band of a carboxypeptidase Y having a molecular weight smaller than that of the bands, to which sugar chains were not added, was observed.

Accordingly, these results clearly show that the human gene MGC3136 compliments the function in budding yeast.

### INDUSTRIAL APPLICABILITY

According to the present invention, the gene of the N-linked sugar chain synthase in human endoplasmic reticulum has been found for the first time. It is known that the deletion or mutation of the gene of the N-linked sugar chain synthase in human endoplasmic reticulum causes congenital disorders of glycosylation syndrome (CDGS). Thus, the gene of the present invention is very useful for diagnosis and treatment for congenital disorders of glycosylation syndrome (CDGS), and the like.

1. A human gene for synthesizing an enzyme catalyzing human N-linked sugar chain synthesis, which is homologous with a gene of an enzyme catalyzing N-linked sugar chain synthesis in yeast endoplasmic reticulum, and is capable of complimenting the function of said gene for a deletion yeast strain of said gene.

2. The human gene according to claim 1, wherein the enzyme catalyzing human N-linked sugar chain synthesis is a glycosyltransferase.

3. A gene which encodes the amino acid sequence represented by SEQ ID NO:2, 4, 6, 8 or 10 or a protein which comprises an amino acid sequence in which one or more amino acids in the amino acid sequence represented by SEQ ID NO:2, 4, 6, 8 or 10 are deleted, substituted or added.

4. The gene according to claim 3, which comprises the nucleotide sequence represented by SEQ ID NO: 1, 3, 5, 7 or 9.

5. A method for diagnosing or treating human congenital disorders of glycosylation syndrome, which comprises using the gene encoding the amino acid sequence according to claim 3 or the gene represented by SEQ ID NO:1, 3, 5, 7 or 9.

6. A recombinant vector which is integrated with a gene selected from the group consisting of:
a human gene for synthesizing an enzyme catalyzing human N-linked sugar chain synthesis, which is homologous with a gene of an enzyme catalyzing N-linked sugar chain synthesis in yeast endoplasmic reticulum, and is capable of complimenting the function of said gene for a deletion yeast strain of said gene; and
a gene which encodes the amino acid sequence represented by SEQ ID NO:2, 4, 6, 8 or 10 or a protein which comprises an amino acid sequence in which one or more amino acids in the amino acid sequence represented by SEQ ID NO:2, 4, 6, 8 or 10 are deleted, substituted or added.

7. A transformant which is transformed by the recombinant vector according to claim 6.

8. A process for producing an enzyme catalyzing human N-linked sugar chain synthesis, which comprises culturing the transformant according to claim 7 in a culture, and collecting the enzyme catalyzing human N-linked sugar chain synthesis from the culture.

9. A method for synthesizing a human N-linked sugar chain, which comprises using the enzyme according to claim 8.
